(19) United States
(12) Patent Application Publication
(10) Pub. No.: US 2011/0238003 A1
(43) Pub. Date: Sep. 29, 2011

(54) METHOD FOR SYSTEMIC DRUG DELIVERY THROUGH THE NAIL

(76) Inventors: Alfredo Emilio Bruno-Raimondi, Biel-Benken (CH); Argeris Jerry Karabelas, Princeton, NJ (US)

(21) Appl. No.: 13/097,896

(22) Filed: Apr. 29, 2011

Related U.S. Application Data
(63) Continuation of application No. 11/746,853, filed on May 10, 2007, now abandoned.
Application No. 11/746,853 is a division of application No. 10/504,175, filed on Aug. 9, 2004, now abandoned.

(30) Foreign Application Priority Data
Feb. 12, 2002 (GB) 0203276.1

(51) Int. Cl. A61M 35/00

(52) U.S. Cl. 604/22

(57) ABSTRACT

A method for systemically delivering a pharmaceutical composition to a human or animal, said method comprising forming an orifice in a nail of a human or animal by means of a laser-based device and applying a pharmaceutical composition in the orifice, wherein said method provides a controlled release of the pharmaceutical composition.

The present invention relates to a method for systemically delivering a pharmaceutical composition to a human or animal, said method comprising forming one or more orifices in a nail of the human or animal by means of a laser-based device and applying a pharmaceutical composition in the orifice.

The nail plate is thick, hard, dense, and represents a barrier for drugs to be able to penetrate in a therapeutically required quantity. Although nail material is similar to the stratum corneum of the skin, being derived from epidermis, it is composed primarily of hard keratin, which is highly disulfide-linked, and is approximately 100-fold thicker than stratum corneum. In order to deliver a sufficient amount of drug into and across the nail plate, the permeability of the nail plate to the drug must be enhanced.

U.S. Pat. No. 6,231,875 describes a method for topical treatment of nail and skin diseases. The patent relates to an acidified composition and methods for increasing the permeability of a nail plate by means of topically applying an acidified composition to the nail plate. U.S. Pat. No. 5,972,317 describes a method for treating diseased nails by topically applying a nail-permeable composition to the nail plate which contains a proteolytic enzyme and a medicament. U.S. Pat. No. 5,181,914 describes a medicating device for human diseased nails and adjacent tissue which contains a viscoelastic gel pad.

U.S. Pat. No. 5,947,956 describes a laser apparatus which is used to make holes in of finger- and toe-nails to apply antifungals to these holes for the treatment of onychomycosis U.S. Pat. No. 4,180,058 describes a method for treating infections of the nail by drilling holes in the nail and placing a caustic-keratolytic agent in the opening to enlarge the opening, and adding a topical therapeutic agent.

The above-mentioned references describe methods or apparatuses for treating nail diseases but none of the references suggests to use the nail, e.g. the healthy nail as drug delivery device to systemically deliver pharmaceutical compositions via nail orifices.

The present invention provides a method for systemically delivering a pharmaceutical composition to the human or animal, said method comprising forming one or more orifices in the nail of the human or animal by means of a laser-based device and applying a pharmaceutical composition in the orifices, wherein said method provides a controlled release of the pharmaceutical composition.

“Orifice” as herein described means any small hole or depression that penetrates 80 to 100% of the nail plate, preferably 90 to 99%.

According to another aspect, the invention provides a method for systemically delivering a pharmaceutical composition to the human or animal, said method comprising forming one or more orifices in the nail, e.g. healthy nail of the human or animal by means of e.g. a laser-based device, applying a pharmaceutical composition in the orifices, and optionally adding a protective layer which prevents the pharmaceutical composition from exiting the outer surface of the orifice and prevents bacteria and dirt from entering the orifice, to the outer surface of the orifices.

In one aspect the method of the invention provides a controlled delayed release, e.g. sustained release, e.g. prolonged release of the pharmaceutical composition that may be used for the continuous treatment of diseases over a period of time.

In a further aspect the method of the invention provides a controlled fast release e.g. immediate release of the pharmaceutical composition. The fast release of pharmaceutical composition may be used to administer pharmaceutical compositions systemically and to avoid a first path effect that may occur by oral administration. The delivery of pharmaceutical composition through the orifice in the nail allows the administration of the pharmaceutical agent directly on the well-perfused nail bed where it enters the blood-stream.

In addition, the method wherein one or more orifices are formed by means of the laser-based device is accomplished with minimum patient discomfort due to the high precision and speed of the laser-based device.

The orifices in the nail are formed preferably by means of the laser-based device comprising a laser which is used to form at least one orifice in the nail. Preferably, numerous orifices are formed in the nail. The orifice may traverse the entire nail or etch the nail depending on the desired mode of treatment and strength of pharmaceutical composition. The diameter of the orifice is preferably from 1 μm (micron) to 1 mm, more preferably from 50 μm (microns) to 200 μm (microns), most preferably from 50 μm (microns) to 100 μm (microns). The orifices preferably are of cylindrical or conical shape.

Typically up to about 500 orifices may be formed in the nail, for example about 50 to about 400, e.g. 100 to 300 orifices.

Any laser may be used provided it is capable of inducing ablation on the nail such as a photoablation laser. Photoablation refers to the melting and explosion of hard tissues. Photoablation is achieved by pulsed laser irradiation of a selected wavelength, power and pulse duration according to the thermal, mechanical and spectral characteristics of the nail of interest. The deposited electromagnetic energy is almost entirely transformed into mechanical energy (i.e. hv≈mv²/2) and the illuminated region is ejected in the form of debris escaping the orifice at ca. 1,000 m/s. In a preferred photoablation process, as the debris removes the deposited energy, the irradiated nail is not heated thus minimizing discomfort.

The laser may be selected from an Erbium (Er):YAG laser, a Nd:YAG laser, a OPO laser, a Ho:YAG laser, a CO₂ laser, a UV laser, or an excimer laser. A suitable UV laser is a nitrogen laser. Suitable excimer lasers include a Kr laser and a Xe laser. Most preferably, the laser is a Er:YAG (λ=2.94 μm) laser, a Ho:YAG laser (λ=2.1 μm), or a CO₂ gas laser (λ=10.6 μm). A combination of lasers may also be used. In a preferred embodiment of the invention, a second laser is used for micromachining the orifice. The ablation temperature is preferably greater than about 100° C.

In one embodiment of the invention one or more small orifices are formed with a single laser shot of ca. 50 μJ of power, ca. 250 μs of duration and the laser system operated at a repetition rate of 3 Hz.

In addition to the laser, the laser-based device may include one or more of the following elements:

(a) a support to secure the nail e.g. by a clamp but leaving the nail plate uncovered;

(b) a computer controlled xyz translation stage module to position the laser beam in the desired area of the nail. Preferably the support (a) may be mounted on this translation stage so that the laser beam is fixed and the toe or finger moves. Alternatively, the toe or finger may be fixed and reflecting elements (e.g. mirrors) are mounted on the translation stage to move the laser beam on the selected parts of the nail plate;

(c) a mirror e.g. dichroic mirror or prism may be used to coaxially mix the laser beams from the laser(s);

(d) an optical focussing element comprising at least one lens;

(e) a computer to monitor the nail plate by means of a video camera or charged coupled device camera which may be used to place the laser beam(s) to the points of the nail plate where orifices are to be formed by means of a computer controlled xyz translation stage (b), and/or to control and/or select the different laser parameters (e.g. the firing of the laser when the desired position of the xyz translation stage (b) has been reached, or the laser power, the pulse duration, or wavelength (e.g. if the laser is a tunable laser);

(f) a video camera or charged coupled device camera to monitor the nail plate on the screen of a personal computer (e);

(g) a feedback sensor (e.g. a photoacoustic sensor made of a piezoelectric material) to ensure that the laser stops after the orifice has reached a predetermined depth (e.g. the nail bed); and

(h) an optical element to multiplex the laser beam(s) (e.g. a diffractive optical element such as Dammann grating) to make more than one orifice (e.g. an array of equally spaced orifices) by a single shot thereby avoiding to make the orifices one-by-one in a subsequent mode.

The pharmaceutical composition of the invention comprises at least one active ingredient. For the purpose of the invention, “active ingredient” means all substances that produce a pharmaceutical or therapeutic effect. Active ingredients may include without limitation photosensitizers, androgens, estrogens, nonsteroidal anti-inflammatory agents, antihypertensive agents, analgesic agents, antidepressants, antibiotics, anticancer agents, anesthetics, antiemetics, antiinfectants, contraceptives, antidiabetic agents, steroids, anti-allergy agents, anti-migraine agents, agents for smoking cessation, and anti-obesity agents.

Examples of active ingredients include the following: acebutolol, acetylcysteine, acetaminophen, acetylsalicylic acid, acyclovir, alprazolam, alfacalcidol, allantoin, allopurinol, aloe vera, ambroxol, amikacin, amiloride, aminoacetic acid, amiodarone, amitriptyline, amlodipine, amoxicillin, ampicillin, ascorbic acid, astemizole, atenolol, beclomethasone, bee propolis, benserazide, benzalkonium hydrochloride, benzocaine, betamethasone, bezafibrate, biotin, biperiden, bisoprolol, bromazepam, bromhexine, bromocriptine, budesonide, bufexamac, buflomedil, bupivacaine, buspirone, caffeine, camphor, captopril, carbamazepine, carbidopa, carboplatin, cefachlor, cefalexin, cefatroxil, cefazolin, cefixime, cefotaxime, ceftazidime, ceftriaxone, cefuroxime, selegiline, chloramphenicol, chlorhexidine, chlor-pheniramine, chlortalidone, choline, cyclosporin, cilastatin, cimetidine, ciprofloxacin, cisapride, cisplatin, clarithromycin, clavulanic acid, clomidine, clomipramine, clonazepam, clonidine, clotrimazole, codeine, cholestyramine, cromoglycic acid, cyanocobalamin, cyproterone, desogestrel, dexamethasone, dexpanthenol, dexamethasone, dextromethorphan, dextropropoxiphen, diazepam, diclofenac, digoxin, dihydrocodeine, dihydroergotamine, dihydroergotoxin, diltiazem, diphenhydramine, dipyridamole, dipyrone, disopyramide, domperidone, dopamine, doxycycline, enalapril, ephedrine, epinephrine, ergocalciferol, ergotamine, erythromycin, estradiol, ethinylestradiol, etoposide, Eucalyptus globulus, famotidine, felodipine, fenofibrate, fenoterol, fentanyl, flavin mononucleotide, fluconazole, flunarizine, fluorouracil, fluoxetine, flurbiprofen, folic acid, folinic acid, furosemide, gallopamil, gemfibrozil, gentamicin, Gingko biloba, glibenclamide, glipizide, clozapine, Glycyrrhiza glabra, griseofulvin, haloperidol, heparin, hyaluronic acid, hydrochlorothiazide, hydrocodone, hydrocortisone, hydromorphone, ipratropium hydroxide, ibuprofen, imipenem, indomethacin, insulin, iohexol, iopamidol, isosorbide dinitrate, isosorbide mononitrate, isotretinoin, ketotifen, ketoconazole, ketoprofen, ketorolac, labetalol, lactulose, levocarnitine, levodopa, levoglutamide, levonorgestrel, levothyroxine, lidocaine, lipase, imipramine, lisinopril, loperamide, lorazepam, lovastatin, medroxyprogesterone, menthol, methotrexate, methyldopa, methylprednisolone, methyltestosterone, metoclopramide, metoprolol, miconazole, midazolam, minocycline, minoxidil, misoprostol, morphine, multivitamin mixtures and combinations and mineral salts, N-methylephedrine, naftidrofuryl, naproxen, neomycin, nicardipine, nicergoline, nicotinamide, nicotine, nicotinic acid, nifedipine, nimodipine, nitrazepam, nitrendipine, nitroglycerine, nizatidine, norethisterone, norfloxacin, norgestrel, nortriptyline, nystatin, ofloxacin, omeprazole, ondansetron, pancreatin, panthenol, pantothenic acid, paracetamol, penicillin G, penicillin V, phenobarbital, pentoxifylline, phenoxymethylpenicillin, phenylephrine, phenylpropanolamine, phenytoin, piroxicam, polymyxin B, povidone-iodine, pravastatin, prazepam, prazosin, prednisolone, prednisone, prilocaine, progesterone, propafenone, propranolol, proxyphylline, pseudoephedrine, pyridoxine, quinidine, ramipril, ranitidine, reserpine, retinol, riboflavin, rifampicin, rutoside, salbutamol, salcatonin, salicylic acid, scopolamine, simvastatin, somatotropin, sotalol, spironolactone, sucralfate, sufentanil, sulbactam, sulfamethoxazole, sulfasalazine, sulpiride, sumatriptan, tamoxifen, tegafur, teprenone, terazosin, terbutaline, terfenadine, testosterone, tetracaine, tetracycline, theophylline, thiamine, ticlopidine, timolol, tranexamic acid, tretinoin, triamcinolone acetonide, triamterene, trimethoprim, troxerutin, uracil, valproic acid, vancomycin, verapamil, vitamin A, vitamin C, vitamin E, and zidovudine. A combination of active ingredients may also be used.

In another aspect the active ingredient of the pharmaceutical composition of the invention may comprise a vaccine. Vaccines may include without limitation Smallpox, Rabies, Plaque, Diphteria, Pertussis, Tuberculosis, Tetanus, Yellow Fever, Injectable Polio Vaccine, Oral Polio Vaccine, Measales, Mumps, Rubella, Hepatitis B, Hepatitis C, Haemophilus influenza Typ B, Japanese Encephalitis, Biomanguinhos, Human Influenza Typ B (Hib), HIV, cancer.

The vaccines are preferably vaccines which require multiple inoculation to achieve protective titers such as Hepatitis B and Hepatitis C.

More preferably the vaccines are vaccines which require long contact with dendritic cells to achieve a cytotoxic T-cell response such as Hepatitis B, HIV, human Papilloma virus (HPV) and cancer. The nail bed has a high concentration of Langerhans cells that stimulate the immune response. Vaccines may be released to the nail bed by the method of the invention. A robust immune response may be obtained by the slow release of vaccines by the method of the invention.

The cancer vaccines may be made of whole cancer cells or of substances contained by the tumor. Preferably the cancer vaccines are selected from the group consisting of whole cancer cells, peptides, proteins, dendritic cells, gangliosides, heat-shock proteins, viral and bacterial vectors and nucleic acids.

The amount of active ingredient in the pharmaceutical composition may vary from 0.1 weight percent, based on the total weight of the pharmaceutical composition, to 100 weight percent. Preferably the active ingredient is present in an amount of from 0.1 to 99, preferably from 20 to 80, more preferably 30 to 70, weight percent, based on the total weight of the pharmaceutical composition. The dose of active ingredient and exposure time depends on the number, diameter and shape of the orifices and on the nature and severeness of the disease to be treated.

Additional components may be used in the pharmaceutical compositions or applied directly to an orifice prior to or following the addition of the pharmaceutical composition to the orifice. Such additional ingredients include natural and/or artificial ingredients which are commonly used to prepare pharmaceutical compositions. Examples of additional ingredients include surfactant (e.g. Aloe Vera), diluents, binders, disintegrating agents, anti caking agents, vitamins, botanicals, supplements, herbs, minerals, trace elements, amino acids (e.g., L. tryptophan), fibers, enzymes, fillers, buffers, colorants, dyes, antioxidants, preservatives, electrolytes, glidants, disintegrates, lubricants, and carrier materials. A combination of additional ingredients may also be used. Such ingredients are known to those skilled in the art.

Following administration of the pharmaceutical composition to the orifice, a protective layer may be placed on the outer surface of the orifice. The protective layer prevents the pharmaceutical composition from exiting the outer surface of the orifice and prevents bacteria and dirt from entering the orifice. Examples of materials useful to form a protective layer include but are not limited to film forming polymers, nail varnish, porcelain, artificial nail, polymer foil, and a patch. It is within the scope of the invention to color-coat the nail whether or not a protective layer is applied.

The pharmaceutical composition may be in the form of a liquid, semi-solid, solid, solution, gel, emulsion, or powder.

The pharmaceutical compositions of the invention are useful for treatment of the known indications of the particular active agent applied. Useful applications include treatment of cancer or age-related macular degeneration (AMD).

In one embodiment of the invention an image of the nail of either the foot or hand is taken. A pattern and the geometry of a suitable array of orifices (e.g. 100 orifices) is calculated e.g. with a software tool. The designed array of orifices traversing the nail is patterned in the nail by laser photoablation. The orifices are filled with the pharmaceutical composition. A nail polish or patch may be used to seal the nail.

In another embodiment of the invention, a laser patterns an array of orifices in a human nail. A surfactant is applied to the orifices e.g. non-ionic surfactant. The orifices are filled with a pharmaceutical composition. A nail polish or patch may be used to seal the nail.

In another embodiment of the invention, a laser patterns an array of orifices in a human nail. The orifices are filled with a photosensitizer. A nail polish or patch is used to seal the nail. The photosensitizer is activated with light. The type of light source including the wavelength and dose may vary depending on the condition to be treated.

While the invention has been described with particular reference to certain embodiments thereof, it will be understood that changes and modifications may be made by those of ordinary skill within the scope and spirit of the following claims.

1. A method for systemically delivering a pharmaceutical composition to a human or animal, said method comprising forming one or more orifices in a nail of the human or animal and applying the pharmaceutical composition in the orifice.

2. A method according to claim 1 wherein the orifices are formed by means of a laser.

3. A method according to claim 1 which provides a controlled release of the pharmaceutical composition.

4. The method according to claim 3 wherein the laser is selected from the group consisting of a Erbium:YAG laser, a Nd:YAG laser, an OPO laser, a Ho:YAG laser, a CO2 laser, a UV laser, an excimer laser, and combinations thereof.

5. The method according to claim 1 wherein a protective layer is placed on the outer surface of the orifice covering the nail.

6. The method according to claim 1 wherein the pharmaceutical composition comprises at least one active ingredient.

7. The method according to claim 6 wherein the active ingredient is a vaccine.

8. The method according to claim 7 wherein the vaccine is selected from the group consisting of Smallpox, Rabies, Plaque, Diphteria, Pertussis, Tuberculosis, Tetanus, Yellow Fever, Injectable Polio Vaccine, Oral Polio Vaccine, Measales, Mumps, Rubella, Hepatitis B, Hepatitis C, Haemophilus influenza Typ B, Japanese Encephalitis, Biomanguinhos, Hib, HIV and cancer.

9. The method according to claim 7 wherein the vaccine requires multiple inoculation to achieve protective titers such as Hepatitis B and Hepatitis C.

10. The method according to claim 7 wherein the vaccine requires long contact with dendritic cells to achieve a cytotoxic T-cell response.

11. The method according to claim 10 wherein the cancer vaccine comprises whole tumor cells or of substances contained by the tumor.

12. The method according to claim 10 wherein the cancer vaccine is selected from the group consisting of whole cancer cells, peptides, proteins, dendritic cells, gangliosids, heat-shock proteins, viral and bacterial vectors and nucleic acids.

13. The method according to claim 6 wherein the active ingredient is selected from the group consisting of a photosensitizer, androgen, estrogen, nonsteroidal anti-inflammatory agent, antihypertensive agent, analgesic agent, antidepressant, antibiotic, anticancer agent, anesthetic, antiemetic, antiinfectant, contraceptive, antidiabetic agent, steroid, anti-allergy agent, anti-migraine agent, agent for smoking cessation, and anti-obesity agent.

14. The method according to claim 6 wherein the active ingredient is selected from the group consisting of acebutolol, acetylcysteine, acetaminophen, acetylsalicylic acid, acyclovir, alprazolam, alfacalcidol, allantoin, allopurinol, aloe vera, ambroxol, amikacin, amiloride, aminoacetic acid, amiodarone, amitriptyline, amlodipine, amoxicillin, ampicillin, ascorbic acid, astemizole, atenolol, beclomethasone, bee propolis, benserazide, benzalkonium hydrochloride, benzocaine, betamethasone, bezafibrate, biotin, biperiden, bisoprolol, bromazepam, bromhexine, bromocriptine, budesonide, bufexamac, buflomedil, bupivacaine, buspirone, caffeine, camphor, captopril, carbamazepine, carbidopa, carboplatin, cefachlor, cefalexin, cefatroxil, cefazolin, cefixime, cefotaxime, ceftazidime, ceftriaxone, cefuroxime, selegiline, chloramphenicol, chlorhexidine, chlor-pheniramine, chlortalidone, choline, cyclosporin, cilastatin, cimetidine, ciprofloxacin, cisapride, cisplatin, clarithromycin, clavulanic acid, clomidine, clomipramine, clonazepam, clonidine, clotrimazole, codeine, cholestyramine, cromoglycic acid, cyanocobalamin, cyproterone, desogestrel, dexamethasone, dexpanthenol, dexamethasone, dextromethorphan, dextropropoxiphen, diazepam, diclofenac, digoxin, dihydrocodeine, dihydroergotamine, dihydroergotoxin, diltiazem, diphenhydramine, dipyridamole, dipyrone, disopyramide, domperidone, dopamine, doxycycline, enalapril, ephedrine, epinephrine, ergocalciferol, ergotamine, erythromycin, estradiol, ethinylestradiol, etoposide, Eucalyptus globulus, famotidine, felodipine, fenofibrate, fenoterol, fentanyl, flavin mononucleotide, fluconazole, flunarizine, fluorouracil, fluoxetine, flurbiprofen, folic acid, folinic acid, furosemide, gallopamil, gemfibrozil, gentamicin, Gingko biloba, glibenclamide, glipizide, clozapine, Glycyrrhiza glabra, griseofulvin, haloperidol, heparin, hyaluronic acid, hydrochlorothiazide, hydrocodone, hydrocortisone, hydromorphone, ipratropium hydroxide, ibuprofen, imipenem, indomethacin, insulin, iohexol, iopamidol, isosorbide dinitrate, isosorbide mononitrate, isotretinoin, ketotifen, ketoconazole, ketoprofen, ketorolac, labetalol, lactulose, levocarnitine, levodopa, levoglutamide, levonorgestrel, levothyroxine, lidocaine, lipase, imipramine, lisinopril, loperamide, lorazepam, lovastatin, medroxyprogesterone, menthol, methotrexate, methyldopa, methylprednisolone, metoclopramide, metoprolol, miconazole, midazolam, minocycline, minoxidil, misoprostol, morphine, multivitamin mixtures and combinations and mineral salts, N-methylephedrine, naftidrofuryl, naproxen, neomycin, nicardipine, nicergoline, nicotinamide, nicotine, nicotinic acid, nifedipine, nimodipine, nitrazepam, nitrendipine, nitroglycerine, nizatidine, norethisterone, norfloxacin, norgestrel, nortriptyline, nystatin, ofloxacin, omeprazole, ondansetron, pancreatin, panthenol, pantothenic acid, paracetamol, penicillin G, penicillin V, phenobarbital, pentoxifylline, phenoxymethylpenicillin, phenylephrine, phenylpropanolamine, phenytoin, piroxicam, polymyxin B, povidone-iodine, pravastatin, prazepam, prazosin, prednisolone, prednisone, prilocaine, progesterone, propafenone, propranolol, proxyphylline, pseudoephedrine, pyridoxine, quinidine, ramipril, ranitidine, reserpine, retinol, riboflavin, rifampicin, rutoside, salbutamol, salcatonin, salicylic acid, scopolamine, simvastatin, somatotropin, sotalol, spironolactone, sucralfate, sufentanil, sulbactam, sulfamethoxazole, sulfasalazine, sulpiride, sumatriptan, tamoxifen, tegafur, teprenone, terazosin, terbutaline, terfenadine, testosterone, tetracaine, tetracycline, theophylline, thiamine, ticlopidine, timolol, tranexamic acid, tretinoin, triamcinolone acetonide, triamterene, trimethoprim, troxerutin, uracil, valproic acid, vancomycin, verapamil, vitamin A, vitamin C, vitamin E, and zidovudine.

15. The method according to claim 1 wherein the pharmaceutical composition is in a form which is selected from the group consisting of a liquid, semi-solid, solid, solution, gel, emulsion, and powder.

16. The method according to claim 6 wherein the active ingredient is present in the pharmaceutical composition in an amount of from 0.1 weight percent, based on the total weight of the pharmaceutical composition, to 100 weight percent.

17. The method according to claim 16 wherein the amount of active ingredient is from 20 to 80 weight percent, based on the total weight of the pharmaceutical composition.

18. The method according to claim 17 wherein the amount of active ingredient is from 30 to 70 weight percent, based on the total weight of the pharmaceutical composition.

19. The method according to claim 1 which is carried out using a laser-based device comprising a laser and at least one element selected from the group consisting of a support, a computer controlled xyz translation stage module, a mirror, an optical focusing element comprising at least one lens, a computer, a video camera, a charged coupled device camera, a feedback sensor, and an optical element.

20. The method according to claim 1 wherein the diameter of the orifice is from 1 μm (micron) to 1 mm.

21. The method according to claim 20 wherein the diameter of the orifice is from 50 μm (microns) to 200 μpm (microns).

22. The method according to claim 21 wherein the diameter of the orifice is from 50 μm (microns) to 100 μm (microns).
